(19) United States
(12) Patent Application Publication
(10) Pub. No.: US 2019/0091400 A1
(43) Pub. Date: Mar. 28, 2019

(54) SYSTEMS AND METHODS FOR WEARABLE TRANSDERMAL DRUG DELIVERY DEVICE

(71) Applicant: Edward Everhart, Farmington, UT (US)

(72) Inventor: Edward Everhart, Farmington, UT (US)

(21) Appl. No.: 16/134,701

(22) Filed: Sep. 18, 2018

Related U.S. Application Data
(60) Provisional application No. 62/562,671, filed on Sep. 25, 2017.

(51) Int. Cl. A61M 5/142

(52) U.S. Cl. CPC A61M 5/14248; A61M 2005/14573

(57) ABSTRACT

A wearable, refillable transdermal drug delivery system is disclosed herein. The system may be generally comprised of a pocket or channel disposed on a garment, and a delivery cartridge containing a predetermined amount of a drug to be delivered. The delivery cartridge may be sized to be frictionally fit in the pocket or channel of the garment.

### BACKGROUND

### 1. The Field of the Invention

The present invention relates to a wearable transdermal drug delivery system and method for use. More particularly, the present invention relates to a wearable, refillable transdermal drug delivery system and method that can be used in conjunction with clothing and the like.

### 2. State of the Art

There are many situations in which it is desirable to have a wearable, refillable transdermal drug delivery system and method for use. Conventional transdermal drug delivery is popular, particularly for use with vitamins, supplements, herbs, and the like.

Transdermal drug delivery has made an important contribution to medical practice. Transdermal delivery systems have continued their steady increase in clinical use for delivery of small, lipophilic, low-dose drugs. Transdermal delivery has a variety of advantages compared with the oral route. In particular, it is used when there is a significant first-pass effect of the liver that can prematurely metabolize drugs. Transdermal delivery also has advantages over hypodermic injections, which are painful, generate dangerous medical waste and pose the risk of disease transmission by needle re-use. In addition, transdermal systems are non-invasive and can be self-administered. They can provide release for long periods of time (up to one week).

However, some issues exist with the use of transdermal patches. Patches for use with exercise to delivery multivitamins and nutritional supplements are growing in popularity. Such patches are typically worn on the arm, etc., while a user is exercising. Adhesives for the patches are prone to decrease effectiveness after prolonged sweating. Another persistent challenge with transdermal drug delivery patches may be to keep the patch in place and in contact with the skin for the desired amount of time. Users may sweat, bathe, and participate in other daily activities that cause a traditional adhesive patch to be ineffective.

Thus, there is a need for a wearable, refillable transdermal drug delivery system and method which can be used with a reliable method of securing the patch that is simple and quick to use, and allows the transdermal drug delivery system to remain in place for prolonged periods of time. It may also be advantageous for the wearable device to be constructed of compression type materials that are popular. It may also be advantageous to provide a method of attaching the transdermal drug delivery system to existing clothing without the need for modification of clothing.

### SUMMARY OF THE INVENTION

An improved wearable, refillable transdermal drug delivery system and method device is provided which allows a user to insert a delivery cartridge carrying a drug to be delivered transdermally into a channel or pocket of a garment which has a void to allow the proximal side of the delivery cartridge to contact the user's dermis.

According to one aspect, the wearable, refillable transdermal drug delivery system and method is deployable for various types of garments such as pants, shorts, armbands, sports compression shorts, knee braces, compression shirts, leggings, sports bras, tank tops, compression running pants, cycling shorts, jerseys, cycling sleeves, headbands, skull caps, etc.

According to another aspect, the delivery cartridge may have a void in the proximal side, and the channel or pocket in which the delivery cartridge is inserted may also have a void in the proximal side such that the delivery cartridge void and the pocket void are substantially overlapping when the delivery cartridge is inserted into the pocket.

According to another aspect, a method is provided for use of the transdermal drug delivery system.

According to another aspect, a device is provided which may attach onto existing clothing, such as via a clip, to provide a wearable transdermal drug delivery device.

### BRIEF DESCRIPTION OF THE DRAWINGS

Various embodiments and features of a wearable, refillable transdermal drug delivery system and method are shown and described in reference to the following numbered drawings:

FIG. 1 shows a perspective view of one configuration of a wearable, refillable, transdermal drug delivery system.

FIG. 2 shows a perspective view of another configuration of a wearable, refillable, transdermal drug delivery system.

FIG. 3 shows a cross-sectional view of a channel or pocket used to hold a delivery cartridge, taken along line 3-3 of FIG. 2.

FIG. 4 shows a cross-sectional view of an exemplary delivery cartridge, taken along line 4-4 of FIG. 2.

FIG. 5 shows a front view of yet another configuration for a delivery cartridge as described herein.

FIG. 6 shows a cross-sectional view of the delivery cartridge of FIG. 5 taken along line 6-6 of FIG. 5.

FIG. 7 shows a perspective view of another configuration of a channel or pocket having a sleeve for mounting.

FIG. 8 shows a cross-sectional view of the pocket of FIG. 7 taken along line 8-8 of FIG. 7.

FIG. 9 shows a front view of another configuration of a drug delivery cartridge according to the present disclosure.

FIG. 10 shows a side view of another configuration of a drug delivery device according to the present disclosure.

FIG. 11 shows a perspective view of the drug delivery device of FIG. 10.

It will be appreciated that the drawings are illustrative and not limiting of the scope of the invention which is defined by the appended claims. The embodiments shown accomplish various aspects of the invention. It is appreciated that it is not possible to clearly show each element and aspect of an invention in a single figure, and as such, multiple figures are presented to separately illustrate the various details of embodiments of wearable, refillable transdermal drug delivery system and method of use in greater clarity. Several aspects from different figures may be used in accordance with a wearable, refillable transdermal drug delivery system in a single structure. Similarly, not every embodiment need accomplish all advantages of various embodiments of a wearable, refillable transdermal drug delivery system and method of use.

### DETAILED DESCRIPTION

The invention and accompanying drawings will now be discussed in reference to the numerals provided therein so as to enable one skilled in the art to practice the present invention. The skilled artisan will understand, however, that the apparatuses, systems and methods described below can be practiced without employing these specific details, or that they can be used for purposes other than those described herein. Indeed, they can be modified and can be used in conjunction with products and techniques known to those of skill in the art in light of the present disclosure. The drawings and descriptions are intended to be exemplary of various aspects of the invention and are not intended to narrow the scope of the appended claims. Furthermore, it will be appreciated that the drawings may show aspects of the invention in isolation and the elements in one figure may be used in conjunction with elements shown in other figures.

Reference in the specification to “one configuration” “one embodiment,” “a configuration” or “an embodiment” means that a particular feature, structure, or characteristic described in connection with the configuration is included in at least one configuration, but is not a requirement that such feature, structure or characteristic be present in any particular configuration unless expressly set forth in the claims as being present. The appearances of the phrase “in one configuration” in various places may not necessarily limit the inclusion of a particular element of the invention to a single configuration, rather the element may be included in other or all configurations discussed herein.

Furthermore, the described features, structures, or characteristics of configurations of the invention may be combined in any suitable manner in one or more configurations. In the following description, numerous specific details are provided, such as examples of products or manufacturing techniques that may be used, to provide a thorough understanding of configurations of the invention. One skilled in the relevant art will recognize, however, that configurations of the invention may be practiced without one or more of the specific details, or with other methods, components, materials, and so forth. In other instances, well-known structures, materials, or operations are not shown or described in detail to avoid obscuring aspects of the invention.

Before the present invention is disclosed and described in detail, it should be understood that the present disclosure is not limited to any particular structures, process steps, or materials discussed or disclosed herein, but is extended to include equivalents thereof as would be recognized by those of ordinarily skill in the relevant art. More specifically, the invention is defined by the terms set forth in the claims. It should also be understood that terminology contained herein is used for the purpose of describing particular aspects of the invention only and is not intended to limit the invention to the aspects or configurations shown unless expressly indicated as such. Likewise, the discussion of any particular aspect of the invention is not to be understood as a requirement that such aspect is required to be present apart from an express inclusion of the aspect in the claims.

It should also be noted that, as used in this specification and the appended claims, singular forms such as “a,” “an,” and “the” may include the plural unless the context clearly dictates otherwise. Thus, for example, reference to “a wearable, refillable transdermal drug delivery system and method” may include one or more of such wearable, refillable transdermal drug delivery system and methods, and reference to “the light” may include reference to one or more of such lights.

As used herein, the term “substantially” refers to the complete or nearly complete extent or degree of an action, characteristic, property, state, structure, item, or result to function as indicated. For example, an object, such as tubing, that is “substantially” enclosed would mean that the object is either completely enclosed or nearly completely enclosed. The exact allowable degree of deviation from absolute completeness may in some cases depend on the specific context, such that enclosing nearly all of the length of a piece of tubing would be substantially enclosed, even if the distal end of the structure enclosing the tubing had a slit or channel formed along a portion thereof. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it lacked a bottom.

As used herein, the term “about” is used to provide flexibility to a numerical range endpoint by providing that a given value may be “a little above” or “a little below” the endpoint while still accomplishing the function associated with the range.

As used herein, a plurality of items, structural elements, compositional elements, and/or materials may be presented in a common list for convenience. However, these lists should be construed as though each member of the list is individually identified as a separate and unique member.

Concentrations, amounts, proportions and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

As used herein, “proximal” refers to a side or portion of a device or system that is disposed closer to the user. “Distal” as used herein refers to a side or portion of a device or system that is farther from the user.

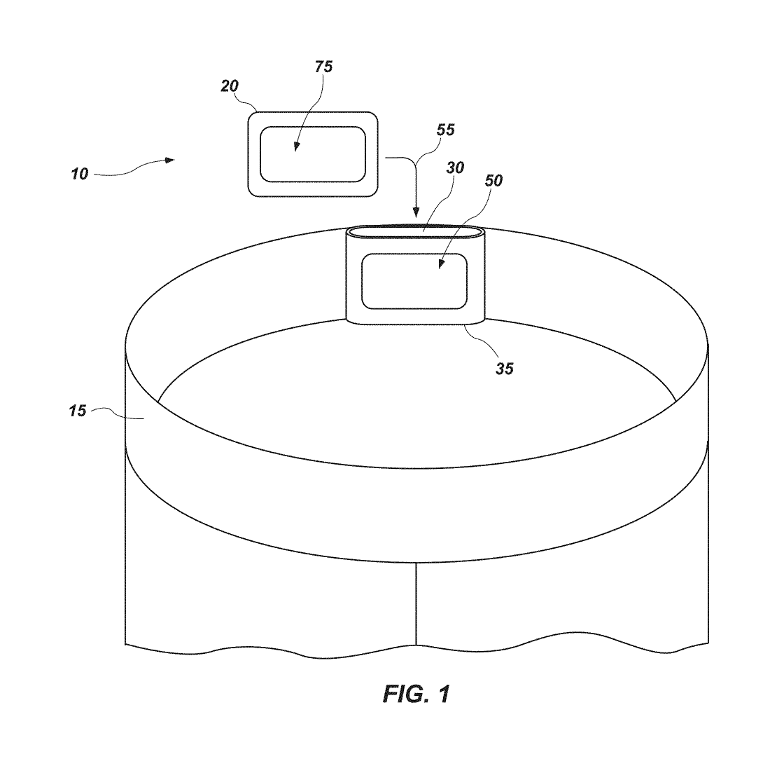

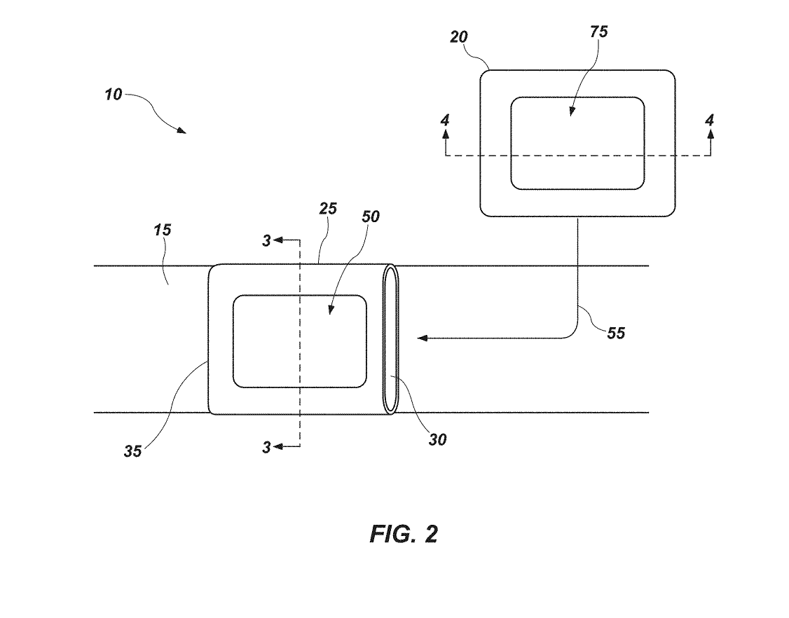

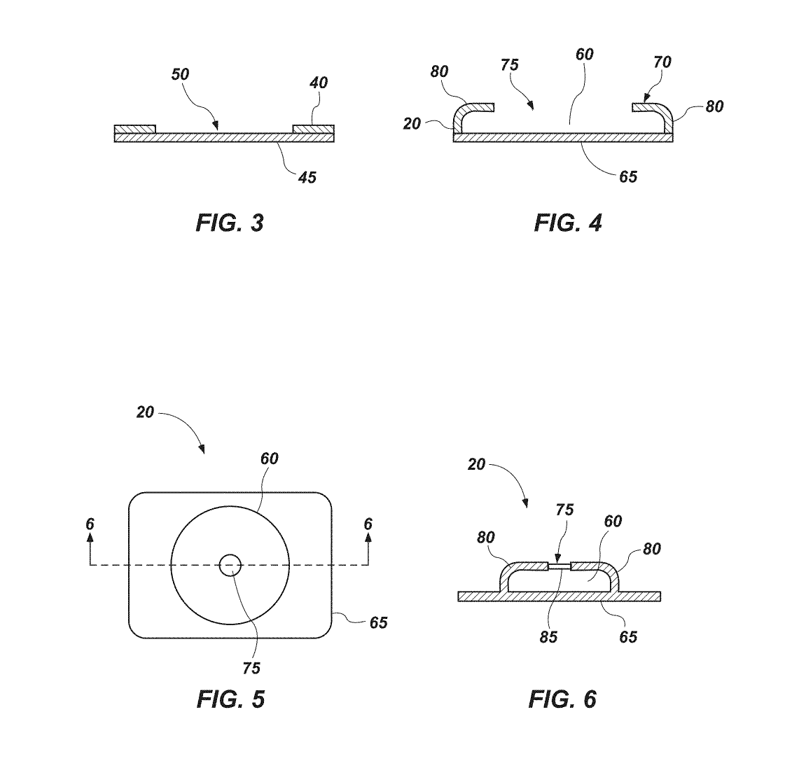

Turning now to FIG. 1 there is shown a wearable, refillable transdermal drug delivery system, generally indicated at 10.The transdermal drug delivery system 10 generally includes a garment 15 and a delivery cartridge 20. The garment may be provided with a channel or pocket 25. The channel or pocket 25 may comprise a first, open end 30 and a second, substantially closed end 35. The channel or pocket 25 also has a proximal side 40, or side that faces the body of a user. (The distal side or backing 45 of the channel or pocket 25 is not visible in FIGS. 1-2, but can be seen in FIG. 3, which shows a cross-sectional view of the channel or pocket 25 taken along line 3-3 of FIG. 2). The proximal side 40 of the pocket 25 defines an opening or void 50. It will be appreciated that the channel or pocket 25 may have any suitable shape and/or size. Similarly, the delivery cartridge 20 may have any suitable shape and/or size. The shape/size shown in the figures is by way of example and not limitation. In some configurations, the garment further comprises a permeable material disposed over the void 50 in the proximal side 40.

The pocket or channel 25 may be generally sized to receive a delivery cartridge 20. The delivery cartridge may be slidably engaged into the channel 25 as indicated by arrow 55. It will be appreciated that the orientation of the pocket or channel 25 with its first, open end 30 will define the direction of insertion for the delivery cartridge 20 (for example, the delivery cartridge may be inserted vertically from the top as indicated in FIG. 1, or horizontally from a side as indicated in FIG. 2). In some configurations, the delivery cartridge 20 may be sized such that it is frictionally fit within the pocket or channel 25, and there is little to no room for the cartridge to move within the pocket or channel 25 (except to be slidably removed). For example, the pocket and the delivery cartridge may be about 1 to 2 inches square.

The delivery cartridge may comprise a reservoir 60, a distal side or backing 65, and a proximal side 70. The proximal side 70 may be formed from one or more reservoir walls 80. Like the pocket or channel 25's proximal side that defines an opening or void 50, the proximal side 70 of the delivery cartridge 20 may define an opening or void 75. Thus, when then delivery cartridge 20 is placed within the pocket or channel 25, the opening or void 50 of the pocket 25 may approximately line up with or coincide with the opening or void 75 of the delivery cartridge 20. This may allow a drug to be delivered to pass from the reservoir of the delivery cartridge, through the void 75 of the delivery cartridge 20, and through the void 50 of the pocket 25 (described in more detail below).

The delivery cartridge 20 may define a reservoir. The reservoir may be any suitable shape and size. The reservoir may be formed by one or more reservoir walls 80 (see FIGS. 4 and 6) attached to the delivery cartridge backing 65 on the distal side. The reservoir walls 80 may be formed of non-permeable material, such as a rigid or semi-rigid plastic. For example, semi-hard polyurethane or another suitable material may be used.

In some configurations, the delivery cartridge 20 (including the backing and reservoir walls) may be formed from a rigid or semi-rigid plastic. The delivery cartridge 20 may be generally shaped to fit within the pocket or channel 25 within the garment 15, for example, rectangular or square, with rounded corners for ease of inserting and removing the delivery cartridge 20 into and out of the pocket 25.

The reservoir of the cartridge may contain a predetermined amount of a drug to be delivered transdermally. The term “drug” is used broadly and encompasses any small molecule suitable for transdermal delivery, including vitamins, nutritional supplements, performance enhancers such as caffeine, other active ingredients, etc. According to some configurations, natural compounds used to benefit energy, skin, mood, pain, immune support, electrolytes, hormones, therapeutics, restorative compounds, regenerative compounds, etc., may be used (such as green tea, turmeric, any kind of vitamins, minerals, herbs, amino acids, botanicals, fatty acids, green coffee beans, DHEA, iodine, THC, cannabis, St. John's wart, etc.). The drug to be delivered may be mixed with other compounds to form a gel mixture. The drug to be delivered may also be mixed with compounds that increase the permeability of the skin's outermost layer in order to increase the efficacy of the transdermal delivery.

In some configurations, the reservoir may be open with no covering over void 75. In other configurations, a permeable material 85 may be placed over void 75 (FIG. 6). The permeable material 85 may allow the drug to be delivered to flow out the void 75 and through the permeable material 85. The permeable material 85 may slow or control the release of the drug to be delivered from the reservoir 60 out the void 75. Similarly, as mentioned above, the void 50 in the garment 15 may also have a permeable material 90 covering it.

According to one configuration, instead of providing the drug to be delivered as a gel in the reservoir, a sponge or other absorbent material may be saturated or substantially saturated in the drug to be delivered and then disposed in the reservoir. PVA, or another water-soluble synthetic polymer, may be used in the reservoir in conjunction with the drug to be delivered. Depending on the desired application and timing of the release of the drug to be delivered, different means of storing the drug to be delivered in the reservoir may be used. For example, for a drug to be delivered more quickly, it may be provided in a gel form in the reservoir. Alternatively, for a drug for which it is desirable to provide a slower delivery over a longer period of time, a sponge or other absorbent material may be substantially saturated with the drug and then placed in the reservoir. In some configurations, it may be desirable to release the drug over around 8 hours.

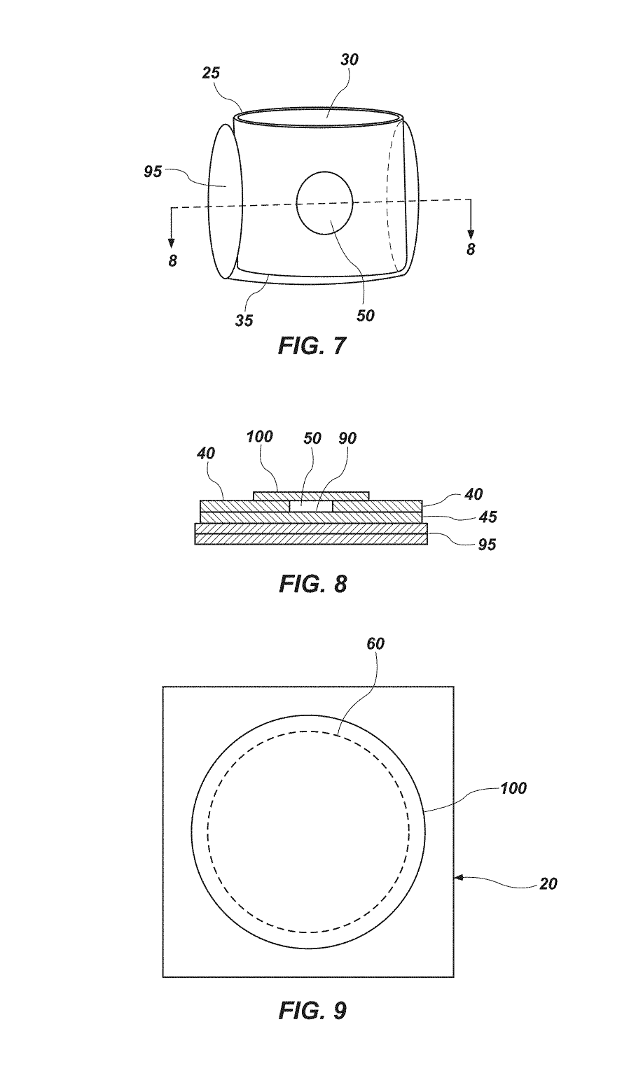

Many types of garments are suitable for use with the present transdermal drug delivery system. For example, in FIGS. 1 and 2, the channel or pocket 25 is disposed on the inside of the back of a waistband on a pair of pants (such as yoga pants). Similarly, the channel or pocket 25 may be disposed on the inside of a shirt sleeve, an armband, the inside of a pair of shorts, etc. FIG. 7 shows a configuration wherein the pocket 25 is provided with a sleeve 95 such that the sleeve may be inserted through an armband (such as a soft, compression-type armband, a metal-type snap-fit armband, etc.). FIG. 8, taken along line 8-8 of FIG. 7, shows the sleeve 95 that may be inserted through an armband, with the pocket 25 comprising a distal side or backing 45 connected to the sleeve 95, and a proximal side 40 defining a void 50 therethrough. The void 50 may be entirely open, or it may be provided with a permeable material 90 covering the void 50. It will be appreciated that this configuration need not be limited to applications for armbands, but may similarly be used for ankle bands, wristbands, leg bands, etc.

According to one aspect, the method of using the drug delivery system herein comprises selecting a garment with the pocket or channel 25, and inserting the delivery cartridge 20 into the pocket or sleeve 25. The delivery cartridge 20 should be inserted such that the void 75 in the reservoir of the delivery cartridge 20 lines up with or coincides with the void or opening 50 of the pocket 25. Thus, a drug to be delivered transdermally may flow from the reservoir 60, through the void 75, through the void 50, and onto the skin of a user.

In some configurations, a cover 100 may be provided to cover the opening or void 75 on the delivery cartridge 20 until the delivery cartridge is ready to be used. In this manner, multiple cartridges may be easy to deploy to end users. When the user desires to use the delivery cartridge 20, he or she may simply remove the covering 100 over the delivery cartridge 20, insert the cartridge 20 into the pocket or sleeve 25 in the garment 15, and then use it to deliver the drugs in the reservoir 60 transdermally. The cover 100 may be any suitable type. In some configurations, the cover 100 may be an adhesive, such that it may be peeled off when the user is ready to insert the cartridge. The cover 100 may be formed of a non-permeable material. According to the method disclosed herein, the cover 100 may be disposed over the void, and then removed upon use.

According to another aspect, the transdermally delivery may be more effective when the pores of a user's skin are open. Thus, the method may include a user exercising or engaging in an activity which causes the user's pores to open (such as use of a steam room or sauna).

According to yet another aspect, a method is disclosed for forming a transdermal, refillable drug delivery cartridge. The delivery cartridge may be formed by any suitable means. According to one configuration, the delivery cartridge may be formed by first disposing a non-permeable cover over the proximal side of the reservoir cap. The non-permeable cover may be, for example, an adhesive cover which may be easily peeled off by the end-user when they desire to use the delivery cartridge. The reservoir may then be filled with a predetermined amount of a drug to be delivered. For example, the drug mixed into a gel may be inserted, or a sponge or other absorbent material that is saturated with the drug to be delivered may be placed in the reservoir. Next, the reservoir cap may be attached to a backing. The method may further include admixing another compound or chemical that increases the permeability of the skin to improve transdermal delivery of the drug.

According to another configuration, an arm band or other garment (such as a garment formed of compression-type material, such as a material comprised of a nylon/spandex blend, a polyester/elastane blend, a nylon/lycra blend, etc.) may be provided to hold one or more drug delivery cartridges 20 in direct contact with the skin of the user. The pocket or channel 25 may be stitched into place in the garment, or it may be held in place, for example, by buttons, zippers, Velcro, etc., such that it may be opened and closed more easily. In some configurations, the compression garment may be provided with a single channel or pocket 25 to hold a drug delivery cartridge 20. In other configurations, the garment may be provided with two or more pockets or channels 25 to hold a drug delivery cartridge 20. For example, a compression arm band with two pockets or channels 25 may allow a user to utilize either two drug delivery cartridges 20 each with half a standard dose, or it may allow a user to utilize two different drug delivery cartridges at a time.

According to another configuration, the delivery cartridge 20 may be provided with a cap or cover 100 over the reservoir that holds the drug to be delivered. The drug delivery cartridge 20 may be first slid into place in the pocket or channel 25 of the garment, and then the cap may be removed. The drug to be delivered may be held in a delivery matrix. For example, polyvinyl alcohol (PVA), or woven/mesh/absorbent materials located in the reservoir, or a gel-type material that holds the drug to be delivered suspended. The drug delivery cartridge, including the backing and the reservoir, may be formed from a polymer based material. This may allow the drug delivery cartridge to have less friction and slide more easily into and out of a pocket or channel 25 of a garment.

The cap 100 on the reservoir may be formed from a film of semi-rigid or rigid polymer. The cap 100 may be glued into place on the drug delivery cartridge, or it may be heat pressed into place. For example, the backing with the reservoir may be formed, the drug to be delivered may be placed into the reservoir (with or without a delivery matrix as described above), and the cap may be glued and/or heat pressed into place.

As shown in FIG. 9, the delivery cartridge 20 may be provided with a backing formed 65 of semi hard polyurethane. The shape and size of the backing may be any suitable size, and may be formed to fit snugly into the channel or pocket 25 of a garment. In FIG. 9, the backing 65 may be approximately square or square, and may have rounded corners or non-rounded corners. For example, in FIG. 9, the backing 65 may be around 2.85 centimeters (1 and ⅛ inches) square, and around 0.158 centimeters ( 1/16 inches) thick. The reservoir 60 (shown in dashed lines in FIG. 9) may be approximately circular, or any other suitable shape. For example, the reservoir may be 1.9 centimeters (¾ inch) across, with a 3 millimeter high wall, and a 0.5 millimeter thick wall formed of soft polyurethane. The cap or cover 100 may be formed of semi-hard poly urethane, and may be approximately 2.54 centimeters (1 inch) across and 0.158 centimeters ( 1/16 inch) thick.

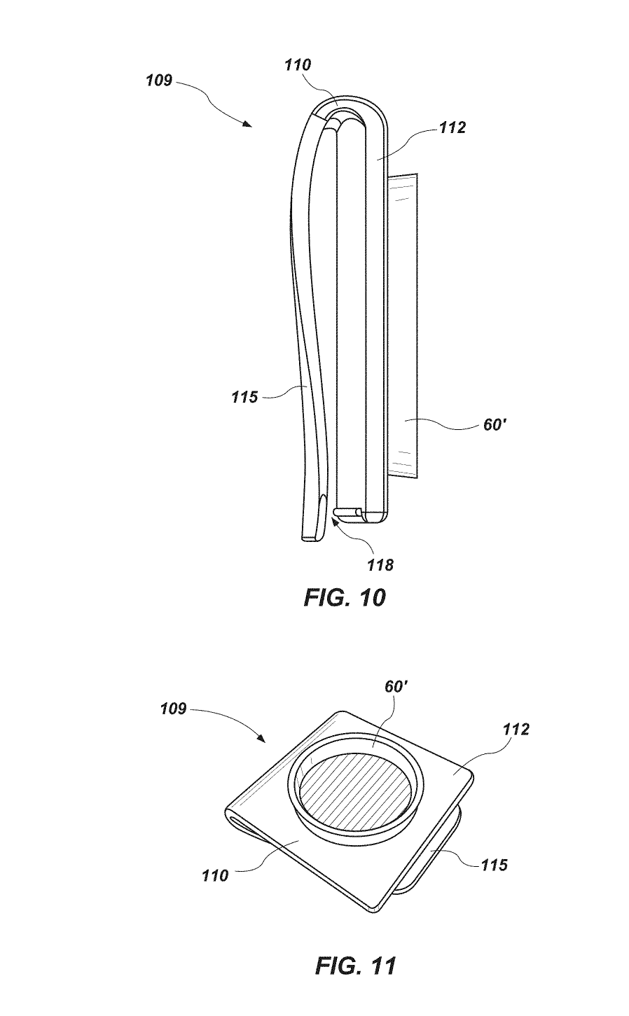

As seen in FIGS. 10-11, another possible configuration for a clip-on delivery cartridge 109 may generally comprise a clip 110, onto which a reservoir 60′ is attached. The clip may have a proximal portion 112, and a distal portion 115. A channel 118 may be formed between the proximal portion 112 and the distal portion 115. This channel 118 may allow a portion of a garment to be placed therein. For example, the channel may be slid onto the waistband of a pair of pants, the ankle band of a pair of pants, the collar of a shirt, etc.

The delivery cartridge 109 may comprise a reservoir 60′ attached to the proximal portion 112. The reservoir 60′ may have an opening or void on the proximal side. When manufactured, a cap or film may be placed over the void in the reservoir 60′. When a user decides to use the clip-on delivery cartridge 109, they may remove the cap or film, and clip the delivery cartridge onto their clothing. Similar to the reservoir described in other configurations, the reservoir 60′ may be formed by one or more reservoir walls attached to the proximal side 112 of the clip 110. The reservoir walls 80 may be formed of non-permeable material, such as a rigid or semi-rigid plastic. For example, semi-hard polyurethane or another suitable material may be used.

Portions of the clip-on delivery cartridge 109, such as portions of the clip 110, reservoir 60′, etc., may be formed from FDA food-grade plastic. Such plastics may include, but are not limited to, Nylon (Tecamid®, Tecast®, Nycast®/Nyoil® FG, Nylatron®), PET-P (Tecapet®, Ertalyte®), Polycarbonate (Makralon®, Tecanat®, Lexan®), ABS (Tecaran®), Noryl® PPO (Tecanyl®), PBT (Polybutylene terephthalate, Hydex® 4101/41010L), Acetal (Tecaform®, Delrin®), Polypropylene (Tecafin®, Proteus®), Polyurethane (Instantroll®), PEEK (Polyetheretherketone, Tecapeek®), UHMW-PE (Ultra-high molecular weight polyethylene, Tivar®), Polyetherimide (Ultem® PEI, Tecapei®, Semitron®), HDPE (High Density Polyethylene, Tecafine®), LDPE (Low Density Polyethylene), Polystyrene High Impact, Polysulfone (Tecason® S), and/or PVDF (Polyvinylidene fluoride, Tecaflon®, Solef®, Kynar®).

In some configurations, the entire clip-on delivery cartridge may be disposable after a single use. In other configurations, the reservoir 60′ may be removable and replaced after each use.

The clip-on delivery cartridge may also be formed using 3-dimensional printing techniques. This may allow the clip-on delivery cartridge to be manufactured relatively simply and inexpensively. For example, the clip portion and reservoir portion may be formed integrally, or may be formed separately and then attached. The reservoir portion may be filled with a drug (such as a vitamin supplement or other drug) to be transdermally delivered, and then have a film or a cap placed over the reservoir. Three-dimensional printing techniques may also allow various portions of the clip-on delivery cartridge to be formed from different materials. Such as the clip portion formed from one type of plastic and the reservoir portion formed from another type of plastic (such as an FDA food-grade plastic as mentioned above).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

Thus there is disclosed a new wearable, refillable transdermal drug delivery system and method device. It will be appreciated that the present disclosure covers multiple inventions which may be used together or separately. Those skilled in the art will appreciate numerous variations which may be made without departing from the scope and spirit of the invention. The appended claims are intended to cover such modifications.

What is claimed is:

1. A refillable transdermal drug delivery system comprising:
a garment comprising channel, the channel having a first, open end and a second, substantially closed end; the channel having a proximal side and a distal side, the proximal side defining a void; and
a delivery cartridge sized to be disposed within the channel of the garment, the delivery cartridge having a reservoir, a distal side and a proximal side, the proximal side defining a void, and the reservoir containing a predetermined amount of a drug to be delivered.

2. The system of claim 1, wherein the delivery cartridge further comprises a permeable material disposed over the void.

3. The system of claim 1, wherein the delivery cartridge further comprises an absorbent material substantially saturated in the predetermined amount of the drug to be delivered, the absorbent material disposed within the reservoir.

4. The system of claim 1, wherein the reservoir is formed by one or more reservoir walls, the reservoir walls comprised of non-permeable material.

5. The system of claim 1, wherein the garment further comprises a permeable material disposed over the void in the proximal side.

6. The system of claim 1, wherein the first, open end is located on a top side of the channel, such that the delivery cartridge may be vertically slidably disposed within the channel.

7. The system of claim 1, wherein the first, open end is located on a right or left side of the channel, such that the delivery cartridge may be slidably engaged horizontally within the channel.

8. The system of claim 1, wherein the garment comprises a pair of pants, the pair of pants having a waistband with an inside, and the channel being disposed on the inside of the waistband.

9. The system of claim 1, wherein the garment comprises an armband, the channel being connected on the distal side to a sleeve, and the sleeve disposed on the armband.

10. The system of claim 1, wherein the predetermined amount of a drug to be delivered comprises a gel mixture containing the predetermined amount of a drug to be delivered.

11. The system of claim 1, wherein the predetermined amount of the drug to be delivered comprises a porous material substantially saturated in the amount of the drug to be delivered.

12. A method for providing a wearable, refillable, transdermal drug delivery system, the method comprising the steps of:
providing a garment, the garment comprising a pocket, the pocket having a first, open end and a second, substantially closed end; the pocket having a proximal side and a distal side, the proximal side defining a void;
providing a delivery cartridge, the delivery cartridge sized to be disposed within the pocket of the garment, the delivery cartridge having a reservoir, a distal side and a proximal side, the proximal side defining a void, and the reservoir containing a predetermined amount of a drug to be delivered;
disposing the delivery cartridge in the pocket of the garment such that the proximal side defining a void of the delivery cartridge substantially overlaps the proximal side defining a void of the pocket.

13. The method of claim 13, wherein the step of disposing the delivery cartridge in the pocket of the garment comprises frictionally fitting the delivery cartridge in the pocket of the garment.

14. The method of claim 13, wherein the step of providing a delivery cartridge comprises providing a delivery cartridge having a non-permeable cover disposed over the void, and the method further comprises the step of removing the non-permeable cover from over the void of the proximal side of the delivery cartridge.

15. The method of claim 13, wherein the method further comprises the step of engaging in an activity which causes skin pores to open.

16. The method of claim 16, wherein the method further comprises the step of exercising.

17. The method of claim 13, wherein the step of providing a delivery cartridge comprises forming a delivery cartridge by disposing a non-permeable cover over the proximal side of a reservoir cap; filling a reservoir with the predetermined amount of a drug to be delivered, and attaching the reservoir cap to a backing.

18. (canceled)

19. A method of manufacturing a wearable, disposable system for transdermal delivery of a drug, the method comprising:
three-dimensionally printing a clip for attachment to a garment from FDA food-grade plastic, the clip comprising a distal side and a proximal side, and a channel formed between the distal side and the proximal side, the channel for receiving a portion of a garment therein; and
three-dimensionally printing a delivery cartridge to be attached to the proximal side of the clip, the delivery cartridge having a reservoir, a distal side and a proximal side, the proximal side defining a void, and the reservoir containing a predetermined amount of a drug to be delivered;
attaching the delivery cartridge to the proximal side of the clip;
filling the delivery cartridge with a drug for transdermal delivery;
covering the void of the proximal side of the reservoir with a removable covering.

20. The method of claim 19, wherein the removable covering comprises at least one of a disposable cap and a disposable film.
